(19) United States
(12) Patent Application Publication
(10) Pub. No.: US 2023/0277199 A1
(43) Pub. Date: Sep. 7, 2023

(54) FLUID-INFUSING ASPIRATION SYSTEM

(71) Applicant: Covidien LP, Mansfield, MA (US)

(72) Inventor: Niall F. Duffy, Galway (IE)

(21) Appl. No.: 18/161,477

(22) Filed: Jan. 30, 2023

Related U.S. Application Data
(60) Provisional application No. 63/268,901, filed on Mar. 4, 2022.

(51) Int. Cl. A61B 17/22; A61M 5/168; A61M 5/142; A61M 1/00

(52) U.S. Cl. CPC A61B 17/22; A61M 5/16804; A61M 5/14212; A61M 1/774; A61B 2017/22079; A61B 2217/007

(57) ABSTRACT

In some examples, a medical system includes a catheter having a proximal end and a distal end, a suction source configured to apply a suction force to the catheter to aspirate a material proximate the distal end of the catheter and from within vasculature of the patient, and a fluid controller configured to control an introduction of a fluid into the catheter between the proximal and distal ends to reduce an amount of blood aspirated from a blood vessel of the vasculature of the patient through the catheter.

This application claims the benefit of U.S. Provisional Patent Application No. 63/268,901, filed on Mar. 4, 2022, and entitled, “FLUID-INFUSING ASPIRATION SYSTEM,” the entire content of which is incorporated herein by reference.

### TECHNICAL FIELD

This disclosure relates to medical devices.

### BACKGROUND

In some cases, medical aspiration can be used to remove material from a patient. For example, medical aspiration can be used to remove a thrombus, such as a clot or other occlusion, from a blood vessel of a patient.

### SUMMARY

This disclosure describes example devices and systems configured to reduce a volume of body fluid (e.g., blood) removed from a patient during a medical aspiration procedure, and related methods. An aspiration catheter can be used to remove a thrombus from a hollow anatomical structure (e.g., a blood vessel) of a patient. For example, a distal opening of the catheter may be positioned in the hollow anatomical structure near a thrombus and a suction force can be applied to a lumen of the aspiration catheter in order to draw the thrombus through the catheter lumen and out of the hollow anatomical structure. In examples described herein, a system is configured to introduce a fluid (e.g., saline) into the catheter to reduce an amount of blood or other body fluid aspirated from the hollow anatomical structure through the catheter.

In some examples, a fluid controller, e.g., under the control of control circuitry, is configured to control an introduction of a fluid into the catheter, e.g., when suction force is not being actively applied to the catheter or when the suction force is being applied to the catheter. For example, the catheter can define an inner lumen through which the thrombus is aspirated and the control circuitry can cause the aspiration system to deliver fluid to fill the inner lumen between the application of suction force to the inner lumen. The delivered fluid can displace at least some body fluid that would ordinarily be removed from the patient via the inner lumen during the application of suction force to the inner lumen. In this way, the delivered fluid can help reduce blood loss during a medical aspiration procedure. In some examples, control circuitry is configured to control at least one of a suction source, a fluid source, or one or more valves cycle to cycle, e.g., alternate or, between applying a suction force to a catheter lumen and supplying fluid to the catheter lumen.

In one example, this disclosure describes a medical system including: a catheter having a proximal end and a distal end and configured to traverse vasculature of a patient; a suction source configured to apply a suction force to the catheter to aspirate a material proximate the distal end of the catheter and from within the vasculature of the patient; and a fluid controller configured to control an introduction of a fluid into the catheter between the proximal and distal ends to reduce an amount of blood aspirated from a blood vessel of the vasculature of the patient through the catheter.

In another example, this disclosure describes a method including: controlling, by control circuitry, a suction source to apply a suction force to a catheter to aspirate a material at a distal opening of the catheter and from within vasculature of a patient; and controlling, by the control circuitry, a fluid control valve to introduce a fluid into the catheter between proximal and distal ends of the catheter to reduce an amount of blood aspirated from a blood vessel of the patient through the catheter.

In another example, this disclosure describes a medical aspiration system including: a catheter defining a catheter lumen; a suction source configured to apply a suction force to the catheter lumen to remove a thrombus from a blood vessel of a patient via the catheter lumen; a fluid source configured to supply a fluid to the catheter lumen in a distal direction to displace blood in the catheter lumen; a fluid control valve configured to control an introduction of a fluid into the catheter lumen; and control circuitry configured to control at least one of the suction source, the fluid source, or the fluid control valve to cycle between applying the suction force to the catheter lumen and introducing the fluid into the catheter lumen, wherein introducing the fluid into the catheter lumen displaces an amount of blood into the blood vessel of the patient and reduces a volume of blood aspirated from the blood vessel through the catheter lumen.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

FIG. 1 is a schematic diagram illustrating an example medical aspiration system.

FIG. 2 is a schematic diagram illustrating another example medical aspiration system.

FIG. 3A is an example timing diagram for controlling aspiration and fluid delivery during a medical procedure.

FIG. 3B is another example timing diagram for controlling aspiration and fluid delivery during a medical procedure.

FIG. 3C is another example timing diagram for controlling aspiration and fluid delivery during a medical procedure.

FIG. 4 is a flow diagram of an example method of cycling aspiration via medical aspiration system of FIG. 1.

### DETAILED DESCRIPTION

The disclosure describes medical aspiration systems configured to reduce an amount of body fluid (e.g., blood) withdrawn from a patient during a medical aspiration procedure, as well as medical devices and methods related to such aspiration systems. A medical aspiration system may be used to treat a variety of conditions, including thrombosis. Thrombosis occurs when a thrombus (e.g., a blood clot or other embolus) forms and obstructs vasculature of a patient. To treat a patient with thrombosis, a clinician may position a medical catheter (also referred to herein as an aspiration catheter) in a hollow anatomical structure (e.g., a blood vessel) of a patient near the thrombus or other occlusion, and apply a suction force (also referred to herein as suction, aspiration force, vacuum force, or negative pressure) to the catheter (e.g., to one or more lumens of the catheter) to engage the thrombus with suction force at a tip of the catheter (e.g., to draw the thrombus through the catheter lumen and out of the hollow anatomical structure). Once the tip of the aspiration catheter has engaged the thrombus, the clinician may remove the aspiration catheter with the thrombus attached to the tip or suction off pieces of the thrombus (or the thrombus as a whole) until the thrombus is removed from the hollow anatomical structure of the patient through a lumen of the aspiration catheter itself and/or through the lumen of an outer catheter in which the aspiration catheter is at least partially positioned. The outer catheter can be, for example, a guide catheter configured to provide additional structural support to the aspiration catheter. In addition, aspiration of thrombus can be performed concurrently with use of a thrombectomy device, such as a thrombus capture system defining a capture basket or the like, to facilitate removal of thrombus via mechanical thrombectomy as well as via aspiration.

The suction force can be generated by any suitable mechanism, such as a vacuum, e.g., by creating a partial vacuum using a vacuum pump, or by direct displacement of fluid in a catheter or tubing via a peristaltic pump. The suction force includes concepts such as suction pressure, vacuum force, vacuum pressure, negative pressure, and the like.

While aspiration in a blood vessel is primarily described herein, the devices, systems, and techniques can be used with other medical procedures for other hollow anatomical structures of a patient.

It may be desirable to limit blood loss during an aspiration procedure. If an aspiration catheter is positioned in a blood vessel and the distal tip of the catheter is not fully engaged with a thrombus as a suction force is being applied to a lumen of the catheter, then a relatively large volume of blood may be unintentionally aspirated from the patient via the catheter. That is, the aspiration system may remove blood and not a thrombus from the patient during some portion of the medical procedure. Given the flow rates through the catheter when the catheter tip is unblocked by a thrombus (i.e., when fluid flow through the catheter is unrestricted), the blood loss rate through the catheter can be relatively high.

During a vacuum based thrombectomy procedure, a clinician may limit the amount of the patient's blood aspirated during the procedure by observing blood flow in a relatively transparent tube of the aspiration system and shutting off the vacuum to the aspiration catheter when the distal end of the catheter is sitting in free blood flow (e.g., not engaged with a thrombus, blockage, clot, or the like). As the procedure progresses the clinician may turn the vacuum on and off periodically to try to aspirate the thrombus, and if the catheter is not at the correct location in close proximity to the thrombus, then the clinician may aspirate blood unintentionally and/or without benefit, e.g., of aspirating a thrombus. The unintentional blood loss can be exasperated with larger bore catheters which have higher flow rates. While this blood loss reduction technique may be useful, it can also be burdensome for the clinician.

According to examples described herein, a medical system is configured to control an introduction of a fluid into an aspiration catheter during an aspiration procedure to reduce an amount of blood aspirated from a blood vessel of the vasculature of the patient through the catheter. The fluid can be introduced automatically, e.g., with minimal or no clinician intervention. In some examples, an aspiration system includes a catheter configured to traverse vasculature of a patient, a suction source configured to apply a suction force to the catheter to aspirate a material proximate a distal end of the catheter (e.g., material at a distal opening, which can be at the distalmost end of the catheter or proximal to but near the distalmost end of the catheter) and from within the vasculature of the patient, and a fluid controller configured to control an introduction of a fluid by a fluid source into the catheter between the proximal and distal ends to reduce an amount of blood aspirated from a blood vessel of the vasculature of the patient through the catheter.

An amount of blood aspirated by the medical system may be indicated by a ratio of blood to fluid aspirated, e.g., collected within a discharge reservoir or evacuation container. In some examples described herein, due to the controlling of the fluid delivery with the application of suction, the medical system is configured to aspirate a blood to fluid ratio of at least 50%, e.g., 50% or greater 75% or greater 90% or greater, 95% or greater, or 100% (e.g., substantially no blood aspirated and 100% of what is aspirated is the fluid and/or material/thrombus).

In some examples, the fluid introduced into the catheter to reduce an amount of blood aspirated includes saline, an antithrombotic agent, saline including an antithrombotic agent, or another suitable biocompatible fluid. The delivery, e.g., introduction, of the fluid through the catheter in the distal direction when the suction source is not actively applying suction to the catheter may help reduce blood loss. For example, the fluid can displace any blood that is present in a lumen of the catheter, and/or tubing or other fluidic components of the system, back into the blood vessel of the patient through the lumen of the catheter, e.g., through the distal end when the catheter is not engaged with a thrombus, blockage, clot, or the like. As another example, the presence of the fluid in the catheter lumen can reduce the amount of blood initially withdrawn through the catheter when suction source next applies the suction force to the lumen of the catheter. In some examples, the medical system is configured to control an amount of suction force applied by the suction source at the distal end of the catheter, e.g., the medical system may include an aspiration valve configured to control an amount of the suction force applied by the suction source at the distal end of the catheter.

In some examples, the medical system includes control circuitry configured to control at least one of the suction source, an aspiration valve, and a fluid controller, e.g., a fluid control valve, a pump, and/or the fluid source, to reduce the amount of blood aspirated from the blood vessel relative to an amount of blood aspirated from the blood vessel without the introduction of the fluid. For example, the control circuitry can be configured to determine whether the catheter is engaged with and/or has captured a material, such as a thrombus (or the like), e.g., via sensing a rate of blood flow through the catheter when the suction source is actively applying a suction force to the lumen of the catheter, via a pressure within the lumen of the catheter and/or other portions of tubing of the aspiration system fluidically connected to the lumen of the catheter, or the any other suitable means for determining whether the catheter has engaged a thrombus. If the control circuitry determines that the distal opening of the catheter has not engaged a thrombus, then the control circuitry may control at least one of the fluid source or fluid control valve to introduce the fluid to the catheter. If the control circuitry determines that the distal opening of the catheter has engaged a thrombus, then the control circuitry may control at least one of the fluid source or the fluid control valve to stop introducing the fluid to the catheter and to aspirate the thrombus.

In some examples, the control circuitry is configured to control the fluid control valve to increase a rate at which the fluid is introduced into the catheter prior to controlling the suction source and/or the aspiration valve to increase the suction force at the distal opening of the catheter. Thus, in addition to reducing an amount of blood aspirated from a blood vessel through the catheter, the fluid delivery can be used to achieve more efficient aspiration.

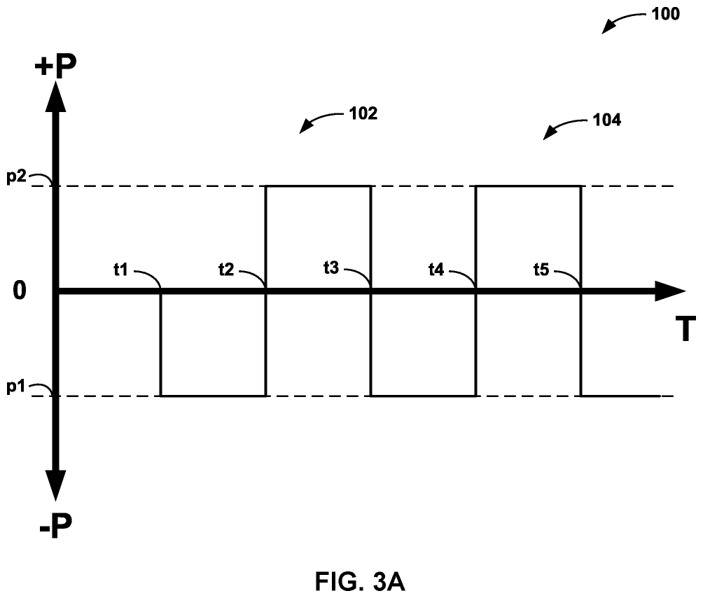

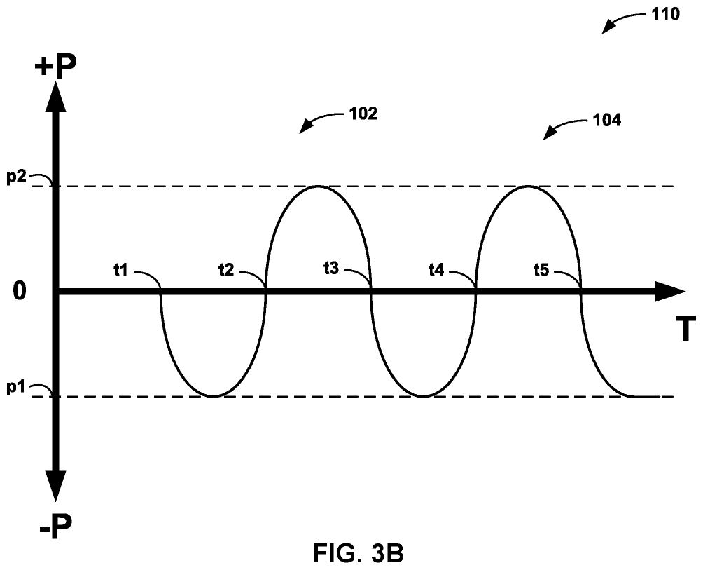

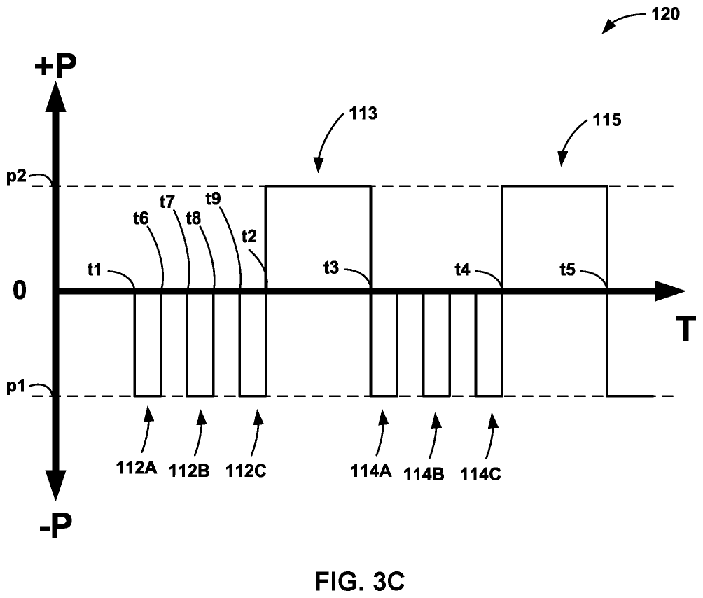

In some examples, the control circuitry is configured to control the suction source, the fluid source, and the fluid control valve to cycle the application of the suction force to the catheter and introducing the fluid to the catheter, e.g., via pulsing and/or alternating aspiration and fluid introduction. As described with reference to FIGS. 3A-3C, cycling can include applying the suction force and delivering the fluid in alternating, but equal time periods or for unequal time periods, as well as via pulses or via a non-pulse pressure waveform. As one example of cycling, the control circuitry may be configured to control at least one of the suction source, the aspiration valve, or the fluid control valve to apply the suction force to the catheter in one or more pulses, and to control at least one of the fluid source or the fluid control valve to introduce the fluid to the catheter between one or more of the pulses. As another example of cycling, the control circuitry may be configured to control at least one of the suction source, the aspiration valve, or the fluid control valve to apply the suction force to the catheter in one or more pulses, and to control at least one of the fluid source or the fluid control valve to introduce the fluid to the catheter during time periods that overlap with one or more of the pulses.

In some examples, the control circuitry is configured to control at least one of the fluid source or the fluid control valve to introduce the fluid to the catheter when the suction source is not actively applying the suction force to the catheter. In other examples, the control circuitry is configured to control at least one of the fluid source or the fluid control valve to introduce the fluid to the catheter when the suction source is actively applying the suction force to the catheter. For example, the control circuitry can control a fluid control valve to cause fluid to be introduced into a lumen of the catheter (in a distal direction) during a relatively low flow aspiration state (a relatively low flow in the proximal direction), and control the control valve to cause less fluid or not fluid to be introduced into the lumen of the catheter (in the distal direction) during a relatively higher flow aspiration state (a relatively higher flow in the proximal direction). The low flow aspiration state can result from, for example, a valve controlling fluid flow through aspiration tubing to be partially closed or fully closed, but configured to permit some fluid through the aspiration tubing even when fully closed.

In some examples, the fluid source includes a pump configured to provide a positive pressure to the fluid (e.g., saline) in a fluid reservoir configured to be fluidically coupled to the catheter and to cause the fluid to flow through the catheter in a distal direction and displace the blood back into the vessel. That is, the fluid source is configured to cause the fluid to flow through a lumen of the catheter in a direction opposite to the direction of fluid flow when the suction source is applying a suction force to the lumen. The medical aspiration system may be configured to remove a first volume of blood from the patient via the catheter, and then to introduce (e.g., supply, deliver) a second volume of the fluid to the catheter, e.g., between one or more of the suction/aspiration pulses. In some examples, the second volume of the fluid may be equal to or greater than the first volume of blood, e.g., so as to displace substantially all of the first volume of blood back into the vessel of the patient.

In some examples, the suction source includes an evacuation container, e.g., a discharge reservoir, fluidically coupled to the pump (or to a second, separate pump) configured to draw the blood through the catheter into the evacuation container. For example, the pump may be configured to provide both a position pressure within a fluid reservoir of the fluid source and a negative pressure, or vacuum, to the evacuation chamber, and other examples, a first pump may be configured to provide a position pressure within a fluid reservoir of the fluid source and a second pump may be configured to provide a negative pressure to the evacuation chamber. Control circuitry may be configured to independently control the first and second pumps, and to thereby independently control the position pressure of the fluid source and thus the pressure, rate, and volume of fluid being introduced into the catheter, and the negative pressure of the suction source and thus the pressure, rate, and volume of blood being aspirated through the catheter.

The pulsing and/or introducing of the fluid may comprise a cycling between aspiration (e.g., applying the suction force to the catheter lumen) and introducing the fluid to the catheter lumen. In some examples, during the first or “aspiration” portion of the cycle the suction source may be fluidically coupled to the catheter and the fluid source may be fluidically disconnected from the catheter, and during the second or “displacement” portion of the cycle, the fluid source may be fluidically coupled to the catheter and the suction source may be fluidically disconnected from the catheter. For example, control circuitry may control a fluid control valve to pulse aspiration and/or introducing the fluid, and/or to cycle between fluidically coupling the suction source/fluidically disconnecting the fluid source and fluidically disconnecting the suction source/fluidically coupling the fluid source.

In some examples, the suction source and/or fluid source may not be completely coupled and/or decoupled, e.g., control circuitry may be configured to control the fluid control valve to be in an intermediate state, e.g., of partially coupling the suction source and the fluid source to the lumen of the catheter. For example, the fluid source may introduce a volume of the fluid at a pressure to both displace blood into the vessel of the patient through the lumen of the catheter and to be aspirated to a discharge reservoir, e.g., so as to flush fluidic tubing and/or components of the aspiration/fluid introducing systems.

In some examples, the medical aspiration system includes one or more one-way valves configured to prevent blood and/or the fluid from flowing to the fluid source, e.g., during aspiration or an aspiration portion of a cycle, or to prevent fluid and/or blood from flowing from the suction source or an aspiration container back through the catheter, e.g., during a displacement portion of a cycle.

The devices, systems, and techniques of this disclosure may provide one or more advantages and benefits. For example, the devices, systems, and techniques may provide reduced blood loss during an aspiration and/or thrombectomy procedure. Additionally, the devices, systems, and techniques may reduce and/or prevent clot formation, e.g., within the catheter and/or other portions of the medical aspiration system, at least because the displacement fluid may help flush out the lumen of the catheter and reduce an amount of stagnant blood in the lumen. Additionally, the devices, systems, and techniques may reduce the burden on a clinician to monitor for clot engagement with the catheter and to react accordingly to turn aspiration on or off.

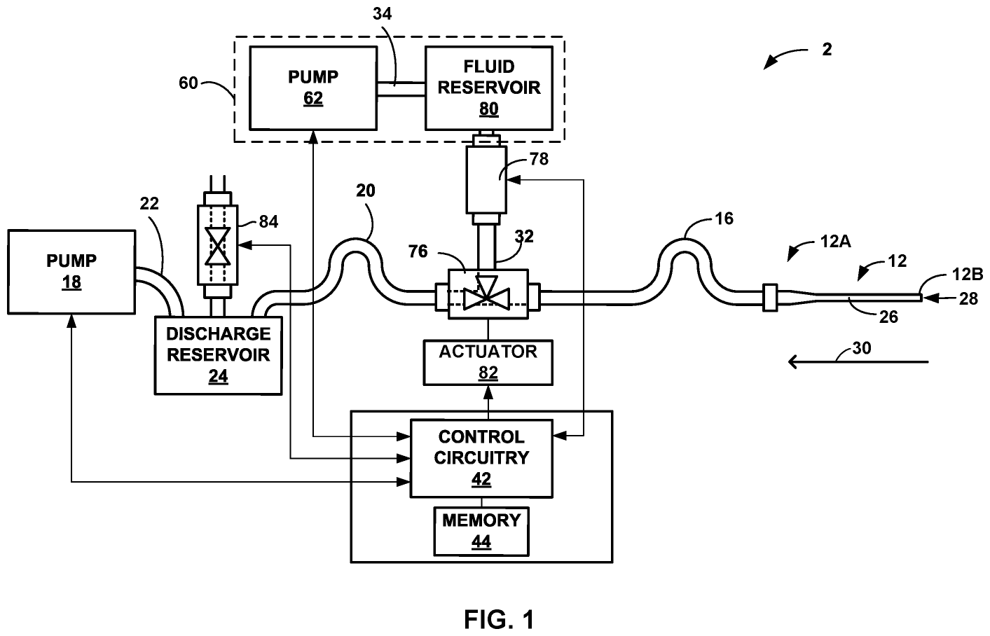

FIG. 1 is a schematic diagram illustrating an example medical system 2 including a pump 18, a discharge reservoir 24, a fluid source 60, valve 76, and a catheter 12. Medical system 2 may be used to treat a variety of conditions, including thrombosis. Thrombosis occurs when a thrombus (e.g., a blood clot or other material such as plaques or foreign bodies) forms and obstructs vasculature of a patient. For example, medical system 2 may be used to treat deep vein thrombosis.

Medical system 2 is configured to remove fluid via catheter 12, e.g., draw fluid from catheter 12 into discharge reservoir 24, via a suction force applied by pump 18 to catheter 12 (e.g., to inner lumen 26 of catheter 12). For example, pump 18 may be a suction source configured to create a negative pressure within inner lumen 26 of catheter 12 to draw a fluid, such as blood, an aspiration fluid, more solid material, or a mixture thereof, in a flow direction 30 (e.g., indicated by the arrow in FIG. 1) and into inner lumen 26 via distal opening 28 of catheter 12. The negative pressure within inner lumen 26 can create a pressure differential between inner lumen 26 and the environment external to at least a distal portion of catheter 12 that causes fluid and other material to be introduced into inner lumen 26 via distal opening 28. For example, the fluid may flow from patient vasculature, into inner lumen 26 via distal opening 28, and subsequently through aspiration tubing 16, valve 76, and aspiration tubing 20 into discharge reservoir 24. Accordingly, the suction source of medical system 2 of FIG. 1 comprises pump 18, an evacuation container in the form of discharge reservoir 24, and a pulsator in the form of valve 76 (also referred to herein as a “fluid control valve” 76).

Catheter 12 and pump 18 may be fluidically coupled using any suitable configuration. In the example shown in FIG. 1, pump 18 is fluidically coupled to catheter 12 via aspiration tubing 22, discharge reservoir 24, aspiration tubing 20, valve 76, and aspiration tubing 16, e.g., aspiration tubing 16 may be fluidically coupled to catheter 12 at proximal end 12A. For example, pump 18 may be coupled to discharge reservoir 24 via aspiration tubing 22, and discharge reservoir 24 can be positioned between pump 18 and catheter 12. In these examples, pump 18 is configured to generate a partial vacuum in discharge reservoir 24 that causes fluid (e.g., blood) and more solid material (e.g., a thrombus) located within an inner lumen 26 of catheter 12 to be drawn into discharge reservoir 24 via aspiration tubing 16, 20 and valve 76. In other examples, pump 18 can be more directly coupled to catheter 12 or may be further fluidically separated from catheter 12 by additional components.

Aspiration tubing 16, 20, 22, as well as other aspiration tubing described herein, is any suitable structure that defines a fluid pathway through which fluid and some relatively small fluid particles may flow between components of medical system 2. The tubing can be formed from any suitable material, such as, but not limited to, polymers, which can be reinforced with bonded, laminated or embedded tubular braids, coils, or other reinforcement member(s).

Catheter 12 is configured to be used as an aspiration catheter to remove a thrombus, such as a clot or other material such as plaques or foreign bodies, from vasculature of a patient. Catheter 12 defines at least one inner lumen, e.g., inner lumen 26 shown in FIG. 1, and at least one distal opening 28 that is open to inner lumen 26. Distal opening 28 may be at a distal-most end of catheter 12 and/or another position along catheter 12, such as in a sidewall of catheter 12 proximal to distal end 12B of catheter 12. Once distal opening 28 of catheter 12 has engaged the thrombus, the clinician may remove catheter 12 with the thrombus held within distal opening 28 (or appended to a distal-most end of an elongated body that defines distal opening 28), or suction off pieces of the thrombus (or the thrombus as a whole) until the thrombus is removed from the blood vessel of the patient, either through inner lumen 26 of catheter 12 itself, and/or through a lumen of an outer catheter (or “sheath,” not shown) in which catheter 12 is at least partially positioned. The outer catheter can be, for example, a guide catheter configured to provide additional structural support to catheter 12.

Catheter 12 includes an elongated body and a hub. The elongated body of catheter 12 is configured to be advanced through vasculature of a patient via a pushing force applied to a proximal portion of the elongated body. Catheter 12 includes any suitable construction for medical aspiration. In some examples, catheter 12 may include an inner liner, an outer jacket, and a structural support member, such as a coil and/or or a braid, positioned between at least a portion of the inner liner and at least a portion of the outer jacket. Catheter 12 may include other structures, such as an expandable member configured to radially expand a distal tip of catheter 12 within a vessel of a patient, e.g., to engage a clot within the vessel.

Catheter 12 is configured to be navigated to any suitable vascular site in a patient. In some examples, catheter 12 is configured to access relatively distal locations in a patient including target tissue sites that may be relatively difficult to reach with a catheter, due at least in part to the tortuous pathway (e.g., comprising relatively sharp twists or turns) through the vasculature to reach these tissue sites. The elongated body of catheter 12 may be structurally configured to be relatively flexible, pushable, and relatively kink- and buckle-resistant, so that it may resist buckling when a pushing force is applied to a relatively proximal section of catheter 12 to advance the elongated body distally through vasculature, and so that it may resist kinking when traversing around a tight turn in the vasculature. In some examples, the elongated body is configured to substantially conform to the curvature of the vasculature. In addition, in some examples, the elongated body has a column strength and flexibility that enables at least the distal portion of the elongated body to be navigated from a femoral artery, through the aorta of the patient, and into the intracranial vascular system of the patient, e.g., to reach a relatively distal treatment site. Alternatively, the elongated body can have a column strength (and/or be otherwise configured) to enable the distal portion of the elongated body to be navigated from a radial artery via an access site in the arm, e.g. at or near the wrist, through the aorta of the patient or otherwise to a common carotid or vertebral artery, and into the intracranial vascular system of the patient, e.g., to reach a relatively distal treatment site.

Although primarily described as being used to reach relatively distal vasculature sites, catheter 12 may also be configured to be used with other target tissue sites. For example, catheter 12 may be used to access tissue sites throughout the coronary and peripheral vasculature, the gastrointestinal tract, the urethra, ureters, fallopian tubes, veins and other body lumens. A length of catheter 12 may depend on the location of the target tissue site within the body of a patient or may depend on the medical procedure for which catheter 12 is used. For example, if catheter 12 is a distal access catheter used to access vasculature in a brain of a patient from a femoral artery access point at the groin of the patient, then an elongated body of catheter 12 may have a working length of about 90 centimeters (cm) to about 145 cm or more, such as about 120 cm, although other lengths may be used (e.g., in the case of a radial access catheter).

As used herein, “suction force” is intended to include, within its scope, related concepts such as suction pressure, vacuum force, vacuum pressure, negative pressure, fluid flow rate, and the like. A suction force can be generated by a vacuum, e.g., by creating a partial vacuum within a sealed volume fluidically connected to a catheter, or by direct displacement of liquid in a catheter or tubing via (e.g.) a peristaltic pump, or otherwise. Accordingly, suction forces or suction as specified herein can be measured, estimated, computed, etc. without need for direct sensing or measurement of force. A “higher,” “greater,” or “larger” (or “lower,” “lesser,” or “smaller”) suction force described herein may refer to the absolute value of the negative pressure generated by the suction source on catheter 12 or another component, such as discharge reservoir 24.

Pump 18 (alternatively “suction source 18”) is configured to create a negative pressure (e.g., vacuum or suction) or otherwise induce fluid flow in inner lumen 26 of catheter 12, e.g., to draw fluid through inner lumen 26 and into discharge reservoir 24. Thus, pump 18 is configured to generate a pressure differential that causes fluid in inner lumen 26 to be drawn out of inner lumen 26 and towards pump 18, e.g., into discharge reservoir 24. For example, pump 18 may include a port configured to couple to aspiration tubing 22, such that the negative pressure created by fluid pump 18 may be applied to the port and through aspiration tubing 22 to a fluid pathway between aspiration tubing 22 and inner lumen 26 of catheter 12. In the example shown in FIG. 1, the fluid pathway further includes discharge reservoir 24, aspiration tubing 16, 20, and valve 76. In an example operation of pump 18, when distal opening 28 of catheter 12 is not blocked, pump 18 may draw fluid from inner lumen 26 of catheter 12 into discharge reservoir 24 through aspiration tubing 16, 20, and through valve 76. As another example, when distal opening 28 is partially or wholly blocked, pump 18 may draw fluid from catheter 12 at a reduced flow rate or, in some instances in which blockage is complete, draw no fluid at all. However, even when distal opening 28 is blocked, pump 18 may be configured to continue to create a vacuum on inner lumen 26 of catheter 12, e.g. via further evacuation of air from discharge reservoir 24. In some examples, pump 18 may be an aspiration pump.

Pump 18 may also be referred to as a fluid pump and can have any suitable configuration. For example, pump 18 (as well as pumps generally within the present disclosure) can include one or more of a positive displacement pump (e.g., a peristaltic pump, a rotary pump, a reciprocating pump, or a linear pump), a centrifugal pump, and the like. In some examples, pump 18 includes a motor driven pump, while in other examples, pump 18 can include a syringe configured to be controlled by control circuitry, and mechanical elements such as linear actuators, stepper motors, etc. As further examples, the pump 18 could comprise a water aspiration venturi or ejector jet. In some examples, pump 18 may be a micromotor pump, a micropump, and in some examples pump 18 may be disposable.

In the example shown in FIG. 1, discharge reservoir 24 includes a relief valve 84, e.g., an aspiration valve, that controls the flow of gases into discharge reservoir 24 from an environment external to discharge reservoir 24. Control circuitry 42 or a clinician may adjust a setting of relief valve 84 in order to control or limit an amount of vacuum pressure developed within discharge reservoir 24 and/or passed through valve 76.

Medical system 2 is configured to supply a fluid to catheter 12, e.g., from fluid source 60 to inner lumen 26. For example, fluid source 60 may be fluidically coupled to inner lumen 26 via valve 76 and aspiration tubing 16, and pump 62 of fluid source 60 may be configured to apply a positive pressure to a fluid in fluid reservoir 80 to cause the fluid to flow in a direction opposite flow direction 30 within inner lumen 26, e.g., a second direction opposite the flow of fluid (blood) during aspiration. That is, fluid source 60 is configured to cause the fluid to flow in a distal direction within catheter 12, e.g., within the same inner lumen 26 through which aspiration is also performed. The fluid within fluid reservoir 80, also referred to herein as a “displacement fluid,” can include saline, saline including one or more antithrombotic agents, an antithrombotic agent, or any suitable fluid configured to displace a blood aspirated by catheter 12 into the vessel of the patient. In some examples, pump 62 is configured to push the displacement fluid via a positive pressure through inner lumen 26 so as to displace the aspirated fluid within valve 76, aspiration tubing 16, and inner lumen 26 back through inner lumen 26 and into the blood vessel via distal opening 28 of catheter 12. This is one example with which an amount of blood aspirated from a blood vessel through catheter 12 can be reduced via introduction of a fluid into catheter 12.

Pump 62 and catheter 12 may be fluidically coupled using any suitable configuration. In the example shown in FIG. 1, pump 62 is fluidically coupled to catheter 12 via tubing 34, fluid reservoir 80, flow restrictor 78, tubing 32, valve 76, and aspiration tubing 16. For example, pump 62 may be coupled to fluid reservoir 80 via tubing 34, and fluid reservoir 80 can be positioned between pump 62 and catheter 12. In these examples, pump 62 is configured to generate a positive pressure in fluid reservoir 80 that causes the displacement fluid to displace aspirated fluid (blood) through inner lumen 26 and into the vessel. In other examples, pump 62 can be more directly coupled to catheter 12 or may be further fluidically separated from catheter 12 by additional components. Tubing 32, 34 may be substantially similar to aspiration tubing 16, 20, 22 described above.

In some examples, pump 62 may be substantially similar to pump 18 described above, except that pump 62 may be configured to create a positive pressure or otherwise induce fluid flow in inner lumen 26 of catheter 12 to displace and/or move aspirated fluid through inner lumen 26 into the vessel. In some examples, pump 62 may be a fluid pump. In some examples, medical system 2 may include a single pump, e.g., pump 18 or pump 62. For example, pump 18 and/or pump 62 may be configured to provide both a negative pressure within discharge reservoir 24 or otherwise apply a suction force to inner lumen 26 and provide a positive pressure within fluid reservoir 80 or otherwise supply the displacement fluid to inner lumen 26. In some examples, pump 18 and/or pump 62 may be configured for bi-directional operation. In some examples, pump 18 may be a micromotor pump, a micropump, and in some examples pump 18 may be disposable.

In the example shown, medical system 2 includes both pump 18 and pump 62 fluidically connected to inner lumen 26 via two different flow paths. In the example shown, catheter 12 and inner lumen 26 are fluidically coupled to each flow path via valve 76. Under the control of control circuitry 42, valve 76 is configured to be movable between at least a first position and a second position. For example, valve 76 can be a two-position three-way valve, such as a three-way ball valve or another suitable valve, e.g., a pinch, poppet, diaphragm, butterfly, slide, or piston valve. In the first position, valve 76 fluidically couples catheter 12 and fluid source reservoir 80, and catheter 12 and pump 18 are not fluidically coupled, e.g., catheter 12 and pump 18 are fluidically disconnected. Thus, in the first position of valve 76, pump 18 does not apply a suction force to inner lumen 26 of catheter 12 and does not draw fluid from inner lumen 26 into discharge reservoir 24. Fluid reservoir 80 can store an incompressible fluid, such as saline. Fluid reservoir 80 may be vented to an external environment. When valve 76 is in its first position, fluid reservoir 80 is fluidically coupled to catheter 12, thereby fluidically connecting pump 62 and fluid reservoir 80 to catheter 12. In some examples, when valve 76 is in its first position, pump 18 is configured to apply a negative pressure to aspiration tubing 22, which creates and/or maintains a negative pressure in discharge reservoir 24. Control circuitry 42 or a clinician can adjust a setting of flow restrictor 78 (when present) in order to adjust a rate of fluid flow of the displacement fluid from fluid source reservoir 80 to catheter 12 through aspiration tubing 16 when valve 76 is in its first position.

In the second position of valve 76, valve 76 fluidically couples pump 18 and discharge reservoir 24 to catheter 12, and pump 18 and fluid source reservoir 80 are not fluidically coupled to catheter 12, e.g., catheter 12 and pump 62 are fluidically disconnected. Thus, in the second position of valve 76, pump 18 (via discharge reservoir 24) can apply a suction force to inner lumen 26 of catheter 12 and draw fluid (blood and/or the displacement fluid) from inner lumen 26 into discharge reservoir 24.

Medical system 2 includes control circuitry 42 configured to control a suction force applied by pump 18 to catheter 12, a positive pressure applied by pump 62 to fluid reservoir 80 to supply the displacement fluid to catheter 12, and valve 76. Fluid control valve 76, e.g., under the control of control circuitry 42, is configured to control an introduction of a fluid into catheter 12 between the proximal and distal ends of catheter 12 to reduce an amount of blood aspirated from a blood vessel through catheter 12. The timing of the fluid delivery relative to the aspiration varies in different examples described herein.

In some examples, control circuitry 42 is configured to control pump 18, pump 62, and valve 76 to cycle between applying the suction force to catheter 12 and delivering the displacement fluid to catheter 12 from fluid source 60. For example, control circuitry 42 may be configured to alternate the application of the suction force to catheter 12 (e.g., to inner lumen 26) and the supplying of the displacement fluid to catheter 12 (e.g., to inner lumen 26). The cycling between the application of the suction force to catheter 12 and the delivery of a displacement fluid to catheter 12 can have any suitable pattern or relative suction/fluid delivery times. For example, control circuitry 42 can control medical system 2 such that the suction force is applied to catheter 12 and the fluid is delivered to catheter 12 for equal amounts of time in a given time period or for unequal amounts of time.

The times during which the suction force is applied to catheter 12 can be referred to as suction periods of the cycle and the time period during which the fluid is delivered to catheter 12 can be referred to as fluid delivery periods. In some examples, a suction period is longer than a fluid delivery period, while in other examples, the fluid delivery period is longer than the suction period. The suction periods and the fluid delivery periods do not overlap in some examples. In these examples, even if pump 18 is not actively apply a suction force to catheter 12, there may be some residual vacuum in inner lumen 26 of catheter 12 due to its length and the time required for the pressure in inner lumen 26 to equalize with the environment external to catheter 12 at distal opening 28. Thus, even when pump 18 is in an off-phase, in which pump 18 is not actively operating to apply a suction force to catheter 12, a negative pressure in inner lumen 26 may still be observed.

In some examples, control circuitry 42 is configured to control pump 18 and valve 76 to apply the suction force to catheter 12 in pulses and to control fluid source 60 and valve 76 to supply the displacement fluid to catheter 12 between the pulses, e.g., when pump 18 is not actively applying the suction force to catheter 12. Control circuitry 42 may be configured to control pump 18 and valve 76 to remove and/or aspirate a first volume of fluid (e.g., blood) from the vessel of the patient via catheter 12 and/or inner lumen 26 during a first portion of a cycle (e.g., a first aspiration pulse) and to control fluid source 60 to supply a second volume of the displacement fluid to catheter 12 during a second portion of a cycle, e.g., before an aspiration pulse of a subsequent cycle.

In some examples, the second volume of displacement fluid is substantially equal (e.g., equal or nearly equal to the extent permitted by manufacturing tolerances) to the first volume of aspirated fluid. In other examples, the second volume of displacement fluid is greater than the first volume of aspirated fluid. In some examples, assuming distal opening 28 is in a fluid filled environment, control circuitry 42 is configured to control pump 18 and valve 76 to aspirate a first volume of fluid that is less than a volume of inner lumen 26, aspiration tubing 16, and valve 76, and to control fluid source 60 and valve 76 to deliver a second volume of displacement fluid is greater than or equal to the first volume, e.g., so as to return substantially all of the aspirated fluid during a pulse or first portion of cycle back to the vessel. In this way, medical system 2 can be configured to reduce the amount of blood that is aspirated into discharge reservoir during a medical aspiration procedure.

Control circuitry 42 can control the cycling between the application of suction force and the delivery of displacement fluid into catheter 12 to displace the suctioned fluid from the immediately prior suction period can repeat as needed until, e.g., a thrombus is engaged with distal opening 28. Thus, rather than aspirating blood into discharge reservoir 24 until the thrombus is engaged with distal opening 28, medical system 2 is configured to periodically deliver the displacement fluid to reduce the amount of blood that is aspirated into discharge reservoir 24. In contrast to systems that merely pulsate the suction force applied to a catheter, medical system 2 further reduces the amount of blood that is aspirated into discharge reservoir 24 via the delivery of the displacement fluid. The delivered displacement fluid is suctioned into discharge reservoir 24 before blood during the next suction period. In some examples, the displacement fluid can also flush out the catheter between suction periods, which can help reduce the accumulation of stagnant blood in the inner lumen 26.

In some examples, valve 76 is configured to operate as a pulsator. For example, control circuitry 42 may be configured to control pump 18 to apply a substantially continuous suction force (e.g., continuous or nearly continuous to the extent permitted by the hardware) to catheter 12 and fluid source 60 to supply a substantially continuous (e.g., continuous or nearly continuous to the extent permitted by the hardware) volume and/or amount of displacement fluid to catheter 12. Control circuitry 42 may be configured to control valve 76 to alternate between first and second positions to pulse the suction force applied to catheter 12 for a period of time and to supply the displacement fluid between the pulses. In other examples control circuitry 42 may be configured to more directly control an operation of pump 18 and fluid source 60 to vary the suction force applied by pump 18 to inner lumen 26 and the displacement fluid supplied to inner lumen 26, e.g. by controlling the motor speed, or stroke length, volume or frequency, or other operating parameters, of pumps 18 and/or 62. In some examples, control circuitry 42 may be configured to coordinate such direct control of pump 18 and fluid source 60 with control of the positioning of valve 76, e.g., to reduce pressure shocks to any of the components of medical system 2 and/or backflow within valve 76, aspiration tubing 16, and/or tubing 32.

Control circuitry 42 may be configured to control a position of valve 76 in order to cycle between applying the suction force to catheter 12 and delivering the displacement fluid to catheter 12 from fluid source 60 using any suitable technique. In the example shown in FIG. 1, valve 76 is actuated between the first and second positions based on an amount of current applied (and/or signal(s) passed) to actuator 82. In some examples, actuator 82 can comprise a solenoid; in such a case, valve 76 can be referred to as a solenoid valve. Actuator 82 can alternatively comprise a linear or rotary actuator, servo, stepper motor, piezoelectric element(s), etc., or any other suitable component(s), or any combination of the foregoing. Control circuitry 72 can control an amount of current applied to (and/or pass signal(s) to) actuator 82 in order to modify a position of valve 76.

Other techniques for modifying a suction force applied by pump 18 to inner lumen 26 of catheter 12 and supplying the displacement fluid to inner lumen 26 of catheter 12 may be used in other examples. For instance, control circuitry 42 may vary the suction force and/or displacement fluid supply by intermittently varying the aspiration force, volume of the displacement fluid, and/or flow rate of the displacement fluid, by periodically varying the aspiration force, volume of the displacement fluid, and/or flow rate of the displacement fluid, or by pulsing the aspiration force, volume of the displacement fluid, and/or flow rate of the displacement fluid, as a few non-limiting examples.

In some examples, control circuitry 42 is configured to control cycling between applying the suction force to catheter 12 and delivering the displacement fluid to catheter 12 based on whether catheter 12 has captured a thrombus, e.g., is engaged with distal opening 28. Control circuitry 42 can determine whether a thrombus is engaged with distal opening 28 using any suitable technique. In some examples, control circuitry 42 receives user input from a clinician, who may determine distal opening 28 is engaged with the thrombus via information provided by medical imaging and provide input to control circuitry 42 indicative of such engagement. Instead of or in addition to the user input, in some examples, control circuitry 42 may determine flow rate of fluid (e.g., blood) within inner lumen 26 or aspiration tubing 16, 20 during application of a suction force to inner lumen 26 based on input from a fluid flow sensor or determine a pressure within inner lumen 26 during application of a suction force to inner lumen 26 based on input from a pressure sensor to determine whether catheter 12 has captured a thrombus. For example, a flow rate of the fluid being aspirated within inner lumen 26 and/or pressure within inner lumen 26 during aspiration without a blockage and/or partial blockage (e.g., without a captured thrombus and/or other material) may be greater than a flow rate and/or pressure within inner lumen 26 with a blockage and/or partial blockage (e.g., with a captured thrombus and/or other material). In some examples, control circuitry 42 may be configured to determine whether catheter 12 has captured a thrombus based on the flow rate of the fluid being aspirated within inner lumen 26 and/or a pressure within inner lumen 26, e.g., by comparison with a threshold flow rate and/or threshold pressure, respectively, as described above.

Instead of or in addition to the user input and/or flow rate, control circuitry 42 may determine whether catheter 12 has captured a thrombus via a physical check and/or measurement, such as via a capacitive and/or optical sensor configured to determine the presence of a thrombus at or within catheter 12.

Thus, in some examples, control circuitry 42 is configured to control cycling (e.g., cause the cycling to occur) between applying the suction force to catheter 12 and delivering the displacement fluid to catheter 12 based on determining that catheter 12 has not captured a thrombus, determining that the flow rate of the fluid being aspirated from the vessel of the patient through catheter 12 (e.g., through inner lumen 26) is greater than or equal to a threshold flow rate, and/or determining that a pressure within catheter 12 (e.g., within inner lumen 26) is equal to or greater than a threshold pressure. The threshold flow rate value and/or the threshold pressure value can be stored in memory 44 of medical system 2 or a memory of another device in communication with control circuitry 42. The cycling may then reduce the amount of fluid (blood) lost by the patient while not aspirating and/or capturing a thrombus. Control circuitry 42 may be configured to cause pump 18, fluid source 60, and/or valve 76 to stop and/or cease cycling between applying the suction force to catheter 12 and delivering the displacement fluid to catheter 12 (e.g., to control pump 18 and valve 76 to apply a suction force to inner lumen 26 to “resume normal or continuous aspiration” to aspirate a fluid and/or thrombus) based on determining that catheter 12 has captured a thrombus, determining that the flow rate of the fluid being aspirated from the vessel of the patient through catheter 12 (e.g., through inner lumen 26) is less than the threshold flow rate, and/or determining that a pressure within catheter 12 (e.g., within inner lumen 26) is less than a threshold pressure. Medical system 2 may then operate to remove the thrombus and/or other material.

Control circuitry 42, as well as other processors, processing circuitry, controllers, control circuitry, and the like, described herein, may include any combination of integrated circuitry, discrete logic circuitry, analog circuitry, such as one or more microprocessors, digital signal processors (DSPs), application specific integrated circuits (ASICs), or field-programmable gate arrays (FPGAs). In some examples, control circuitry 42 may include multiple components, such as any combination of one or more microprocessors, one or more DSPs, one or more ASICs, or one or more FPGAs, as well as other discrete or integrated logic circuitry, and/or analog circuitry. In some examples, control circuitry 42 is or includes a “smart” device or system, including, but not limited to, a robotic device (e.g., a robotic surgical system), a device configured to operate with the aid of artificial intelligence (AI), a virtual reality (VR) system configured to aid a clinician with the medical procedure, cloud-based interfaces for data processing and/or data storage, or any combination thereof.

Memory 44 may store program instructions, such as software, which may include one or more program modules, which are executable by control circuitry 42. When executed by control circuitry 42, such program instructions may cause control circuitry 42 to provide the functionality ascribed to control circuitry 42 herein. The program instructions may be embodied in software and/or firmware. Memory 44 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, ferroelectric RAM (FRAM), or any other digital media.

In some examples, but not all examples, a distal portion of catheter 12 includes an expandable distal portion (not shown) configured to expand radially outward to widen distal opening 28 for engaging with a thrombus. The expanded distal opening 28 may help increase the suction force applied to a thrombus.

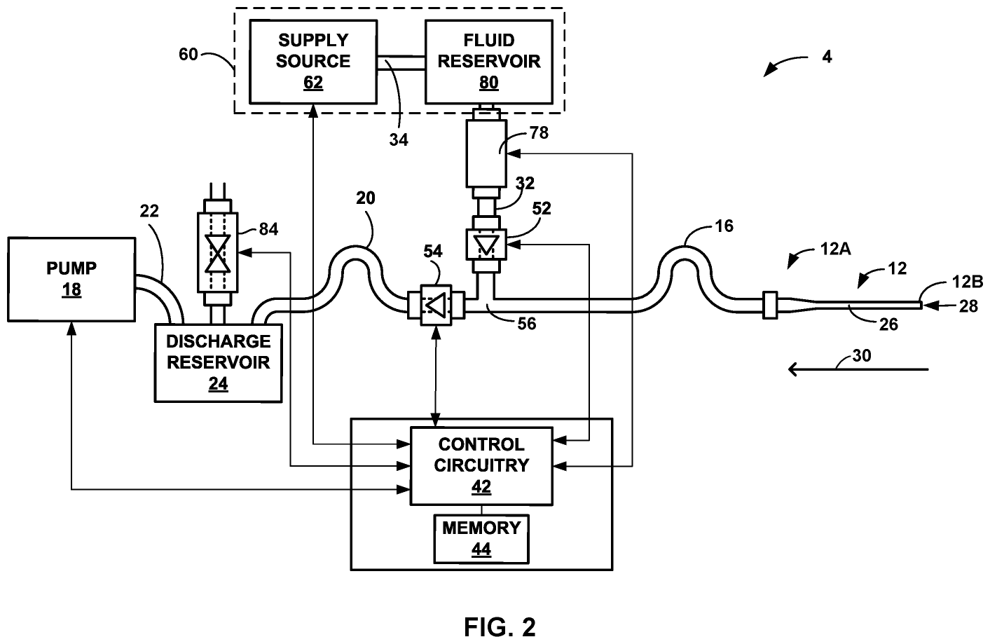

FIG. 2 is a schematic diagram illustrating another example medical system 4 including a pump 18, a discharge reservoir 24, a fluid source 60, valves 52, 54, and a catheter 12. Medical system 4 may be substantially the same as medical system 2 described above, except that medical system 4 includes valves 52, 54 rather than valve 76. Medical system 4 may be used to treat a variety of conditions, including thrombosis.

Valve 52 is configured to help reduce or even prevent a flow of a fluid (e.g., blood, the displacement fluid, or a mixture of blood and the displacement fluid) from aspiration tubing 16 to fluid source 60, e.g., backflow through tubing 32. During normal aspiration and/or a first portion of a cycle when pump 18 is applying a suction force to inner lumen 26, it may be desirable to prevent the aspirated fluid from the vessel from entering the displacement fluid supply flow path, e.g., the flow path including pump 62, fluid reservoir 80, tubing 32, valve 52, and optionally flow restrictor 78. Valve 52 is configured to prevent blood from entering and/or dwelling within the fluid supply flow path. Valve 52 may be a one-way valve.

Valve 54 is configured to prevent a flow of a fluid (e.g., blood, the displacement fluid, or a mixture of blood and the displacement fluid) from aspiration tubing 20 to aspiration tubing 16, e.g., backflow through aspiration tubing 16. During a second portion of a cycle when pump 62 is supplying the displacement fluid to inner lumen 26, there may be a pressure differential at the junction 56 between the aspiration flow path and the fluid supply flow path that may draw a portion of aspirated fluid (e.g., blood, saline, or a mixture thereof) to flow towards inner lumen 26. It may be desirable to prevent the aspirated fluid within the aspiration flow path, e.g., pump 18, aspiration tubing 22, discharge reservoir 24, and/or aspiration tubing 20, from flowing towards inner lumen 26. Valve 54 is configured to prevent aspirated fluid from flowing from the aspiration flow path towards inner lumen 26. Valve 54 may be a one-way valve.

Control circuitry 42 may be configured to control pump 18 and fluid source 60 to cycle between applying the suction force to catheter 12 and delivering the displacement fluid to catheter 12. For example, control circuitry 42 may be configured to coordinate and/or synchronize the cycling and/or alternating between suction and supply via the pump 18 and pump 62 in the absence of valve 76.

FIG. 3A is a plot 100 of an example timing diagram for pulsing and/or cycling aspiration using a medical aspiration system. For example, control circuitry 42 of medical system 2 and/or medical system 4 may be configured to cycle between applying the suction force to catheter 12 and delivering the displacement fluid to catheter 12 according to plot 100. In some examples, control circuitry 42 of medical system 2 and/or medical system 4 are configured to introduce the displacement fluid to catheter 12 between aspiration pulses.

In some examples, plot 100 is a plot of a pressure within inner lumen 26 of catheter 12 versus time during cycling. In other examples, plot 100 is a plot of the set-point pressures control circuitry 42 is configured to actualize within inner lumen 26, e.g., the design pressures that control circuitry 42 is configured to achieve via controlling pump 18, pump 62, and/or valve 76.

In the example shown, plot 100 includes two cycles, e.g., a first cycle 102 from time t1 to time t3, and a second cycle 104 from time t3 to time t5. Alternatively, plot 100 may represent a first aspiration pulse from time t1 to time t2, a second aspiration pulse from time t3 to time t4, and introducing the displacement fluid between the first and second aspiration pulses from time t2 to time t3 and between the second aspiration pulse and a subsequent aspiration pulse (not shown) from time t4 to t5. Plot 100 may include more or fewer cycles, e.g., one cycle, a partial cycle such as a half of a cycle, or three or more full or partial cycles, or more or fewer aspiration pulses with or without introduction of the displacement fluid between one or more of the aspiration pulses (e.g., the pressure in the lumen would be substantially near zero between aspiration pulses without introduction of the displacement fluid). As noted above, in some examples, control circuitry 42 is configured to initiate and cease cycling and/or alternating pulsing/fluid introduction based on a determination of whether catheter 12 has captured a thrombus or whether too much blood is being aspirated, e.g., based on flow rate through inner lumen 26, an amount of blood collected within discharge reservoir, based on user input, or the like. In some examples, control circuitry 42 is configured to initiate and cease cycling based on a predetermined amount of time or number of cycles, e.g., when searching and/or drawing in a thrombus for aspiration from a vessel of the patient.

In the example shown, the period of the first cycle 102 is the difference between t3 and t1, and the period of the second cycle 104 is the difference between t5 and t3. In some examples, first cycle 102 and second cycle 104 may have the same period, and in other examples first cycle 102 and second cycle 104 may have different periods. In some examples, plot 100 may include a plurality of cycles each of which may have the same period, or all of which may have different periods, or any combination thereof.

In the example shown, first cycle 102 and second cycle 104 each have a first portion of the cycle in which the pressure is negative, e.g., −p1 or a first aspiration pulse, corresponding to pump 18 applying a suction force to inner lumen 26 for a period of time from t1 to t2 and from t3 to t4, respectively. Each of first cycle 102 and second cycle 104 each have a second portion of a cycle in which the pressure is positive, e.g., +p2, corresponding to fluid source 60 supplying the displacement fluid to inner lumen 26 for a period of time from t2 to t3 and from t4 to t5, respectively. In some examples, p1 and p2 may have the same magnitude, and in other examples p1 and p2 may have different magnitudes.

In some examples, first cycle 102 and second cycle 104 (or first aspiration pulse and second aspiration pulse) may each have the same negative pressure p1 during the first portion and the same position pressure p2 during the second portion (e.g., between aspiration pulses), as shown, or the magnitudes of the negative and positive pressures of each of cycles 102 and 104 may be different from each other. For example, control circuitry 42 may be configured to dynamically control pump 18, pump 62, and/or valve 76 to dynamically control the amplitude, period, and duty cycle of plot 100. In the example shown, the amplitude of plot 100 is p1 subtracted from p2 (or, alternatively and equivalently, the addition of the magnitudes of p1 and p2), the duty cycle of first cycle 102 is the ratio of the time the cycle is at negative pressure to positive pressure, e.g., (t2−t1)/(t3−t1), and similarly the duty cycle of second cycle 104 is the ratio of the time the cycle is at negative pressure to positive pressure, e.g., (t4−t3)/(t5−t3). Control circuitry 42 may be configured to dynamically change any or all of the amplitudes, periods, and duty cycles of each cycle when controlling cycling of a medical system.

In some examples, control circuitry 42 controls pump 18 and/or valve 76 to aspirate a first volume of fluid, e.g., blood, by controlling the amplitude (p1, e.g., causing the suction force within inner lumen 26) and the time of the first portion of a cycle, e.g., t2−t1 of cycle 102. In some examples, control circuitry 42 may control pump 18 such that the amplitude and time of the first portion of cycle 102 such that the first volume is less than or equal to a volume of inner lumen 26 and aspiration tubing 16, e.g., such that all of the aspirated fluid is within aspiration tubing 16 and/or inner lumen 26 between junction 56 and distal opening 28 and available to by pushed back into the vessel during the subsequent second portion of the cycle.

Control circuitry 42 may control pump 62, pump 18, and/or valve 76 to deliver a second volume of displacement fluid, e.g., saline, by controlling the amplitude (p2, e.g., caused by delivering the displacement fluid via a positive pressure within fluid reservoir 80 or any other suitable method) and the time of the second portion of a cycle, e.g., t3−t2 of cycle 102. In some examples, control circuitry 42 may control the amplitude and time of the second portion of cycle 102 such that the second volume of displacement fluid displaces at least a portion (e.g., all or just a portion) of the first volume aspirated fluid back into the vessel through distal opening 28. In some examples, control circuitry 42 controls pump 62 and/or valve 76 to control the amplitude and time of the second portion of cycle 102 such that the second volume of displacement fluid is greater than or equal to the first volume and displaces substantially all the first volume aspirated fluid back into the vessel through distal opening 38. In some examples, a portion of the second volume of the displacement fluid may also flow through distal opening 28 and into the vessel. In some examples, the second volume of displacement fluid may include, or may comprise, an antithrombotic agent, and a portion of the antithrombotic agent may be within aspiration tubing 16, inner lumen 26, and/or flow through distal opening 28 into the vessel.

In the example shown in FIG. 3A, plot 100 includes cycles 102 and 104 (or aspiration pulses and displacement fluid introduction periods) that cycle and/or pulse aspiration according to a rectangular, or square wave, cycling and/or set point curve. The level of the plot 100 can correspond to the amount of suction provided by the system or the amount of fluid flow that is allowed by the system. For instance, the plot 100 can represent the relative amount that a controllable valve is opened, or the relative strength of suction provided by a pump. For example, medical system 2 or 4 may be configured to change the pressure within the lumen of catheter 12 relatively quickly, e.g., via switching valve 76. In other examples, medical system 2 or 4 may be configured to change the pressure within the lumen according to a different functional shape, e.g., a triangle wave, a sine wave, or any suitable functional shape. FIG. 3B is a plot 110 of an example timing diagram for pulsing and/or cycling aspiration and fluid delivery according to a sinusoidal-like timing. For example, plot 100 may be a set-point for the pressures within the lumen of catheter 102 and plot 110 may be the corresponding pressures actualized within the lumen of catheter 102, e.g., the response time of medical system 2 or 4 may include a delay in cycling/pulsing/switching pressures by design (e.g., so as to avoid “shocking” aspiration tubing, valves, catheter 12, or any other components of medical system 2 or 4) or by the limits of the response time of components of medical system 2 or 4, e.g., pumps 18, 62, and/or valve 76.

In some examples, control circuitry 42 controls pump 62, pump 18, and/or valve 76 to cycle and/or pulse aspiration according to any suitable periodic or non-periodic timing. For example, control circuitry 42 may control pump 62, pump 18, and/or valve 76 to cycle according to timing diagram 120 of FIG. 3C. FIG. 3C is a plot 120 of an example timing diagram for cycling aspiration and fluid delivery using a medical aspiration system described herein. Plot 120 includes a first plurality of aspiration pulses 112A, 112B, and 112C (collectively “aspiration pulses 112”), a first displacement fluid introduction time period 113, a second plurality of aspiration pulses 114A, 114B, and 114C (collectively “aspiration pulses 114”), and a second displacement fluid introduction time period 115. In some examples, aspiration pulses 112 is a first portion of a first cycle, time period 113 is a second portion of the first cycle, aspiration pulses 114 is a first portion of a second cycle, and time period 115 is a second portion of the second cycle. Although shown with three pulses, in other examples aspiration pulses 112 and 114 may include more or fewer pulses, e.g., two pulses or four or more pulses.

In some examples, medical system 2 or 4 may not completely shut off suction and/or aspiration between aspiration pulses and/or displacement fluid introduction. For example, plot 120 may not be at zero pressure between aspiration pulses 112 or 114, e.g., the pressure of plot 120 may be negative between zero and −p1 between times t6 and t7 and between times t8 and t9. Such implementations can allow the system to operate in a low-flow mode, where the amount of fluid flow is significantly reduced instead of completely the stopped.

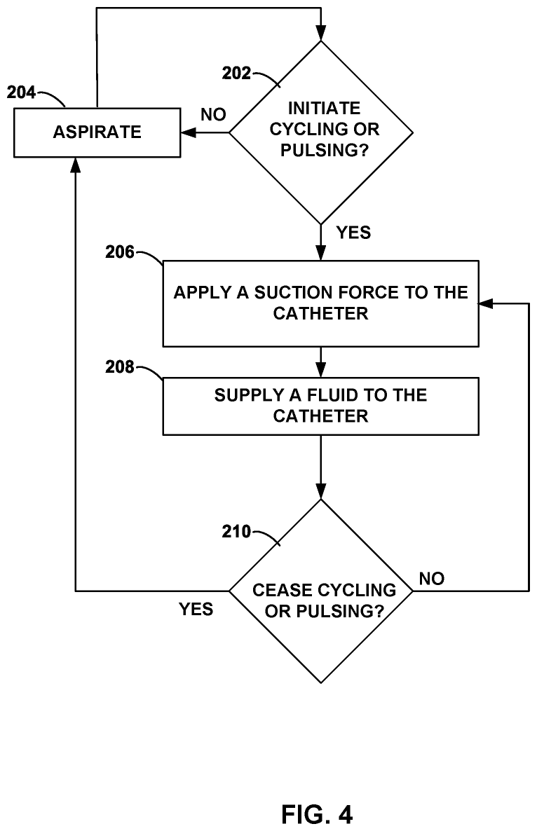

In some examples, control circuitry 42 may control medical system 2 and/or medical system 4 to cycle between applying a suction force to a catheter and delivering the displacement fluid to a catheter from a fluid source. FIG. 4 is a flow diagram of an example method of cycling aspiration via a medical aspiration system. While FIG. 4 is described with reference to medical systems 2, 4, and control circuitry 42, in other examples, the technique may be performed by another system, alone or in combination with medical systems 2, 4. Although described in terms of cycling and/or pulsing aspiration, in some examples the cycling may be and/or include pulsing aspiration.

As shown in FIG. 4, control circuitry 42 determines whether to initiate cycling, and/or pulsing aspiration, between applying a suction force to catheter 12 and delivering the displacement fluid to catheter 12 from a fluid source 60 (202). For example, control circuitry may determine whether to initiate cycling and/or pulsing based on whether catheter 12 has captured a thrombus, e.g., as indicated by user input, a flow rate (in a proximal direction towards pump 18) within inner lumen 26, a pressure within inner lumen 26, or the like or combinations thereof. If control circuitry 42 determines that catheter 12 has captured a thrombus, if a flow rate within inner lumen 26 is less than or equal to a threshold flow rate, or if a pressure within inner lumen 26 is less than or equal to a pressure threshold, then control circuitry 42 controls medical system 2, 4 to aspirate (204) a fluid, thrombus, and/or material from the vessel of the patient at (204), e.g., the “NO” branch at step (202).

If control circuitry 42 determines that catheter 12 has not captured a thrombus, if a flow rate within inner lumen 26 is greater than or equal to a predetermined threshold flow rate, or if a pressure within inner lumen 26 is greater than or equal to a pressure threshold, then control circuitry 42 may control medical system 2, 4 to initiate cycling and/or pulsing, e.g., the “YES” branch at step (202).

Control circuitry 42 controls medical system 2, 4 to apply a suction force to inner lumen 26 of catheter 12 (206). For example, control circuitry 42 may control pump 18 to apply the suction force for a first portion of a cycle 102, 104. In some examples, the first portion of the cycle 102, 104 may be a suction (e.g., aspiration) pulse, and control circuitry 42 may control pump 18 to apply the suction force to inner lumen 26 via one or more suction pulses. Control circuitry 42 may control pump 18 to draw in fluid (e.g., blood) through inner lumen 26 in flow direction 30, e.g., from distal opening 28 to remove blood from the vessel of the patient. Control circuitry 42 may control pump 18 to remove a first volume of blood from the vessel of the patient during the first portion of the cycle (or during a first pulse or first plurality of pulses), and in some examples the first volume is less than a volume of inner lumen 26 and aspiration tubing 16.

In some examples, as in medical system 2, control circuitry 42 may control valve 76 and/or actuator 82 to control the position of valve 76 to be in a first position fluidically coupling pump 18 and catheter 12 and (inner lumen 26) and not fluidically coupling fluid source 60 and catheter 12 (and inner lumen 26). In some examples, as in medical system 4, control circuitry 42 may aspirate a fluid (blood, the displacement fluid, or a mixture thereof) through a one-way valve (e.g., valve 54) fluidically coupled between pump 18 and catheter 12 and configured to prevent the fluid from flowing to catheter 12 from pump 18, discharge reservoir 24, and/or aspiration tubing 20 fluidically coupled between valve 54 and fluid discharge reservoir 24 and/or pump 18.

Control circuitry 42 controls medical system 2, 4 to deliver the displacement fluid to catheter 12 and/or inner lumen 26 from fluid source 60 (208). For example, control circuitry 42 may control pump 62 to supply the displacement fluid to inner lumen 26 for a second portion of the cycle 102, 104, e.g., between aspiration pulses. In some examples, the displacement fluid may comprise saline, an antithrombotic agent, a thrombotic agent within saline, or any suitable displacement fluid. In some examples, control circuitry 42 may control fluid source 60 to alternate application of the suction force to catheter 12 and supplying the displacement fluid to catheter 12, e.g., between suction pulses and/or when pump 18 is not actively applying the suction force to catheter 12. In some examples, control circuitry 42 controls fluid source 60 to deliver the displacement fluid to inner lumen 26 in a flow direction (e.g., a distal direction) opposite flow direction 30 (e.g., a proximal direction). In some examples, control circuitry 42 controls fluid source 60 to deliver a second volume of displacement fluid during the second portion of the cycle 102, 104, and the second volume may be equal to or greater than the first volume of fluid aspirated during the first portion of the cycle 102, 104. The second volume of displacement fluid may displace a portion of, or all of, the first volume of aspirated fluid in the flow direction opposite flow direction 30, e.g., into the vessel through distal opening 28.

In some examples, as in medical system 2, control circuitry 42 controls medical system 2, 4 to deliver the displacement fluid to catheter 12 by at least controlling valve 76 and/or actuator 82 to control the position of valve 76 to be in a second position fluidically coupling fluid source 60 and catheter 12 and (inner lumen 26) and not fluidically coupling pump 18 and catheter 12 (and inner lumen 26). In some examples, as in medical system 4, a one-way valve (e.g., valve 52) fluidically coupled between fluid source 60 and catheter 12 may prevent at least one of the first fluid, the second fluid, or a mixture of the first and second fluids from flowing to fluid source 60.

In some examples, control circuitry 42 controls pump 18, fluid source 60, and/or valve 76 to alternate application of the suction force to catheter 12 and supplying the displacement fluid to catheter 12. For example, control circuitry 42 may control pump 18 and pump 62 to alternate application of the suction force to catheter 12 and supplying the displacement fluid to catheter 12. In other examples, control circuitry 42 may control pump 18 and pump 62 to continuously provide a suction force (e.g., negative pressure, vacuum, etc.) and a positive pressure to the displacement fluid and/or fluid source reservoir, respectively, and control valve 76 to cycle between the first position fluidically coupling the pump 18 and catheter 12 and fluidically disconnecting fluid source 60 and catheter 12 and a second position fluidically coupling fluid source 60 and catheter 12 and fluidically disconnecting pump 18 and catheter 12.

Control circuitry 42 controls medical system 2, 4 determines whether to cease cycling the suction force applied to catheter 12 and delivering the displacement fluid to catheter 12 (210). For example, control circuitry 42 controls medical system 2, 4 may determine whether to cease cycling based on at least one of catheter 12 having captured a thrombus, the flow rate of the fluid (e.g., blood) through catheter 12 being less than or equal to a threshold flow rate, or the pressure within catheter 12 being less than or equal to a threshold pressure, or the like. In some examples, a flow rate of less than a threshold flow rate, or a pressure of less than a threshold pressure, may indicate that distal opening 28 is engaged with a thrombus, and the thrombus may at least partially block or impede the flow of the fluid into, and within, inner lumen 26. For example, if the flow rate within inner lumen 26 is reduced (e.g., due to a blockage of distal opening 28), the pressure within inner lumen 26 may drop or reduce, e.g., due to the suction force. If control circuitry 42 determines that catheter 12 has captured a thrombus, if a flow rate within inner lumen 26 is less than or equal to a threshold flow rate, or if a pressure within inner lumen 26 is less than or equal to a pressure threshold, then control circuitry 42 may control medical system 2, 4 to cease cycling and commence aspiration at (204), e.g., the “YES” branch at step (210).

If control circuitry 42 determines that catheter 12 has not captured a thrombus, if a flow rate within inner lumen 26 is at least the threshold flow rate, or if a pressure within inner lumen 26 is less than a pressure threshold, then control circuitry 42 may control medical system 2, 4 to continue cycling, e.g., the “NO” branch at step (210).

In some examples, control circuitry 42 may determine that catheter 12 no longer has captured a thrombus or the thrombus has been aspirate, that a flow rate within inner lumen 26 is at least the threshold flow rate, or that a pressure within inner lumen 26 is less than a pressure threshold during aspiration at (204). The method may then follow the “YES” brand at step (202) and control circuitry 42 may initiate cycling (206), (208) as described above.

In one or more examples, the functions described in this disclosure, e.g., with respect to control circuitry 42 or any of the systems described herein, may be implemented in hardware, software, firmware, or any combination thereof. If implemented in software, the functions may be stored on, as one or more instructions or code, a computer-readable medium and executed by a hardware-based processing unit. The computer-readable medium may be an article of manufacture including a non-transitory computer-readable storage medium encoded with instructions. Instructions embedded or encoded in an article of manufacture including a non-transitory computer-readable storage medium encoded, may cause one or more programmable processors, or other processors, to implement one or more of the techniques described herein, such as when instructions included or encoded in the non-transitory computer-readable storage medium are executed by the one or more processors. Example non-transitory computer-readable storage media may include RAM, ROM, PROM, EPROM, EEPROM, FRAM, flash memory, a hard disk, a CD-ROM, a floppy disk, a cassette, magnetic media, optical media, or any other computer readable storage devices or tangible computer readable media.

In some examples, a computer-readable storage medium comprises non-transitory medium. The term “non-transitory” may indicate that the storage medium is not embodied in a carrier wave or a propagated signal. In certain examples, a non-transitory storage medium may store data that can, over time, change (e.g., in RAM or cache).

The functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements.

The present disclosure includes the following non-limiting examples.

Example 1: A medical system including: a catheter having a proximal end and a distal end and configured to traverse vasculature of a patient; a suction source configured to apply a suction force to the catheter to aspirate a material proximate the distal end of the catheter and from within the vasculature of the patient; and a fluid controller (e.g., a fluid control valve) configured to control an introduction of a fluid into the catheter between the proximal and distal ends to reduce an amount of blood aspirated from a blood vessel of the vasculature of the patient through the catheter.

Example 2: The medical system of example 1, wherein the system is configured to displace blood aspirated into the catheter through an opening at or near the distal end by the introduction of the fluid into the catheter.

Example 3: The medical system of example 1 or example 2, wherein the system is configured to control an amount of the suction force applied by the suction source at a distal opening of the catheter.

Example 4: The medical system of example 3, further comprising control circuitry configured to control the suction force at the distal opening of the catheter and the fluid controller to reduce the amount of blood aspirated from the blood vessel relative to an amount of blood aspirated from the blood vessel without the introduction of the fluid.

Example 5: The medical system of example 4, wherein the control circuitry is configured to control the fluid controller to increase a rate at which the fluid is introduced into the catheter prior to controlling the aspiration valve to increase the suction force at a distal opening of the catheter and further comprising an aspiration valve configured to control the amount of the suction force applied by the suction source at the distal opening of the catheter.

Example 6: The medical system of any one of examples 1-5, wherein the suction source is configured to apply the suction force to a lumen of the catheter, the medical system further comprising a fluid source configured to introduce the fluid into the lumen via the fluid controller.

Example 7: The medical system of example 6, further comprising control circuitry configured to control the suction source, the fluid source, and the fluid controller to alternate the application of the suction force to the catheter and the introduction of the fluid into the lumen.

Example 8: The medical system of example 6 or example 7, further comprising control circuitry configured to control at least one of the suction source or an aspiration valve to apply the suction force to the catheter in one or more pulses, and wherein the control circuitry is configured to control at least one of the fluid source or the fluid controller to introduce the fluid to the catheter between one or more of the one or more pulses.

Example 9: The medical system of any one of examples 1-8, wherein the control circuitry is configured to control the fluid controller to introduce the fluid to the catheter when the suction source is not actively applying the suction force to the catheter.

Example 10: The medical system of any one of examples 1-9, wherein the control circuitry is configured to control the fluid controller to introduce the fluid to the catheter when the suction source is actively applying the suction force to the catheter.

Example 11: The medical system of any one of examples 6-10, wherein the suction source is configured to aspirate a thrombus through a lumen of the catheter in a first flow direction, and wherein the fluid source is configured to introduce the fluid to the lumen in a second flow direction opposite the first flow direction.

Example 12: The medical system of any one of examples 6-11, further comprising control circuitry configured to control at least one of the suction source or an aspiration valve to apply the suction force to the catheter in one or more pulses, wherein the suction force is configured to remove a first volume of blood from the patient via the catheter during the one or more pulses, and wherein the control circuitry is configured to control at least one of the fluid source or the fluid controller to introduce a second volume of the fluid to the catheter between one or more of the one or more pulses.

Example 13: The medical system of example 12, wherein the second volume is equal to or greater than the first volume.

Example 14: The medical system of example 12 or example 13, wherein the second volume of the fluid displaces the first volume of the blood in the second flow direction.

Example 15: The medical system of any one of examples 6-14, wherein the fluid source comprises a fluid pump configured to provide a positive pressure to the fluid in a fluid reservoir configured to be fluidically coupled to the catheter.

Example 16: The medical system of any one of examples 1-15, wherein the suction source comprises an evacuation container fluidically coupled to an aspiration pump, and wherein the aspiration pump is configured to draw the blood through the catheter into the evacuation container.

Example 17: The medical system of any one of examples 6-16, further comprising a first one-way valve configured to prevent at least one of the fluid or the blood from flowing to the fluid source.

Example 18: The medical system of any one of examples 4-16, wherein the control circuitry is configured to introduce the fluid to the catheter based on at least one of determining that the catheter has not captured a thrombus, determining a flow rate of a body fluid from the patient through the catheter is greater than or equal to a threshold flow rate, or determining a pressure within the catheter is equal to or greater than a threshold pressure.

Example 19: The medical system of any one of examples 4-18, wherein the control circuitry is configured to control the fluid controller to stop introducing the fluid to the catheter based on at least one of determining that the catheter has captured a thrombus, determining a flow rate of a body fluid from the patient through the catheter that is less than a threshold flow rate, or determining a pressure within the catheter is less than a threshold pressure.

Example 20: The medical system of any one of examples 1-19, wherein the fluid comprises at least one of saline or an antithrombotic agent.

Example 21: A method including: controlling, by control circuitry, a suction source to apply a suction force to a catheter to aspirate a material at a distal opening of the catheter and from within vasculature of a patient; and controlling, by the control circuitry, a fluid controller (e.g., a fluid control valve) to introduce a fluid into the catheter between proximal and distal ends of the catheter to reduce an amount of blood aspirated from a blood vessel of the patient through the catheter.

Example 22: The method of example 21, wherein controlling the fluid controller to introduce the fluid comprises introducing the fluid into the catheter to displace blood aspirated into the catheter through an opening at or near the distal end of the catheter.

Example 23: The method of example 21 or example 22, wherein controlling the suction source comprises controlling, by the control circuitry, an aspiration valve to control an amount of the suction force applied by the suction source at the distal opening of the catheter.

Example 24: The method of example 23, wherein controlling the aspiration valve and the fluid controller reduces the amount of blood aspirated from the blood vessel relative to an amount of blood aspirated from the blood vessel without the introduction of the fluid.

Example 25: The method of example 24, further comprising controlling, by the control circuitry, the fluid controller to increase a rate at which the fluid is introduced into the catheter prior to controlling the aspiration valve to increase the suction force at the distal opening of the catheter.

Example 26: The method of any one of examples 21-25, wherein controlling the suction force to apply the suction force to the catheter comprises controlling the suction force to apply the suction force to a lumen of the catheter, and wherein controlling the fluid controller to introduce the fluid into the catheter comprises controlling a fluid source to introduce the fluid into the lumen via the fluid controller.

Example 27: The method of example 26, further comprising controlling, by the control circuitry, the suction source, the fluid source, and the fluid controller to alternate the application of the suction force to the catheter and introducing the fluid to the catheter.

Example 28: The method of example 26 or example 27, further comprising controlling, by the control circuitry, at least one of the suction source or an aspiration valve to apply the suction force to the catheter in one or more pulses, and wherein the control circuitry is configured to control at least one of the fluid source or the fluid controller to introduce the fluid to the catheter between one or more of the one or more pulses.

Example 29: The method of any one of examples 26-28, wherein the fluid source comprises a first pump configured to provide a positive pressure to the fluid in a fluid reservoir configured to be fluidically coupled to the catheter.

Example 30: The method of any one of examples 21-29, wherein controlling the fluid controller to introduce the fluid into the catheter comprises controlling the fluid controller to introduce the fluid into the catheter when the suction source is not actively applying the suction force to the catheter.

Example 31: The method of any one of examples 21-29, wherein controlling the fluid control valve to introduce the fluid into the catheter comprises controlling the fluid controller to introduce the fluid into the catheter when the suction source is actively applying the suction force to the catheter.

Example 32: The method of any one of examples 21-31, wherein controlling the suction source to apply the suction force to the catheter comprises controlling the suction force to aspirate a thrombus through a lumen of the catheter in a first flow direction, and wherein controlling the fluid controller to introduce the fluid into the catheter comprises controlling the control circuitry to introduce the fluid into the lumen in a second flow direction opposite the first flow direction.

Example 33: The method of any one of examples 21-32, wherein controlling the suction source to apply the suction force to the catheter comprises controlling the suction force to remove a first volume of blood from the patient via the catheter during the one or more pulses, and wherein controlling the fluid controller to introduce the fluid into the catheter comprises controlling the fluid source to introduce a second volume of the fluid between one or more of the one or more pulses.

Example 34: The method of example 33, wherein the second volume is equal to or greater than the first volume.

Example 35: The method of example 32 or example 34, wherein the second volume of the fluid displaces the first volume of the blood in the second flow direction.

Example 36: The method of any one of examples 21-35, wherein the suction source comprises an evacuation container fluidically coupled to an aspiration pump, and wherein the second pump is configured to draw the blood through the catheter into the evacuation container.

Example 37: The method of any one of examples 21-36, further includes determining, by the control circuitry, that at least one of the catheter has not captured a thrombus, a flow rate of a body fluid from the patient through the catheter is greater than or equal to a threshold flow rate, or a pressure within the catheter is greater than or equal to a threshold pressure; and controlling, by the control circuitry, the fluid controller to introduce the fluid based on determining that at least one of the catheter has not captured the thrombus, the flow rate of the body fluid through the catheter is greater than or equal to the threshold flow rate, or the pressure within the catheter is greater than or equal to the threshold pressure.

Example 38: The method of any one of examples 21-37, further includes determining, by the control circuitry, that at least one of the catheter has captured a thrombus, a flow rate of a body fluid from the patient through the catheter is less than a threshold flow rate, or a pressure within the catheter is less than a threshold pressure; and controlling, by the control circuitry, the fluid controller to introduce the fluid based on determining that at least one of the catheter has captured the thrombus, the flow rate of the body fluid through the catheter is less than the threshold flow rate, or the pressure within the catheter is less than the threshold pressure.

Example 39: The method of any one of examples 21-38, wherein the fluid comprises at least one of saline or an antithrombotic agent.

Example 40: A medical aspiration system including: a catheter defining a catheter lumen; a suction source configured to apply a suction force to the catheter lumen to remove a thrombus from a blood vessel of a patient via the catheter lumen; a fluid source configured to supply a fluid to the catheter lumen in a distal direction to displace blood in the catheter lumen; a fluid control valve configured to control an introduction of a fluid into the catheter lumen; and control circuitry configured to control at least one of the suction source, the fluid source, or the fluid control valve to cycle between applying the suction force to the catheter lumen and introducing the fluid into the catheter lumen, wherein introducing the fluid into the catheter lumen displaces an amount of blood into the blood vessel of the patient and reduces a volume of blood aspirated from the blood vessel through the catheter lumen.

Example 41: The medical aspiration system of example 40, wherein the control circuitry is configured to control the fluid control valve to increase a rate at which the fluid is introduced into the catheter prior to controlling the aspiration valve to increase the suction force at the distal opening of the catheter.

Example 42: The medical aspiration system of any one of examples 40 and 41 or any of examples 40 and 41, wherein the control circuitry is configured to control the at least one of the suction source, the fluid source, or the fluid control valve to cycle between applying the suction force to the catheter lumen and introducing the fluid into the catheter lumen by at least controlling at least one of the suction source, the fluid source, or the fluid control valve to alternate the application of the suction force to the catheter lumen and the introduction of the fluid into the catheter lumen.

Example 43: The medical aspiration system of any one of examples 40 through 42 or any of examples 40 through 42, wherein the control circuitry is configured to control the at least one of the suction source, the fluid source, or the fluid control valve to cycle between applying the suction force to the catheter lumen and introducing the fluid into the catheter lumen by at least controlling the suction source or an aspiration valve to apply the suction force to the catheter in one or more pulses and controlling at least one of the fluid source or the fluid control valve to introduce the fluid to the catheter between one or more of the one or more pulses.

Example 44: The medical aspiration system of any one of examples 40-43, wherein the control circuitry is configured to control at least one of the fluid source or the fluid control valve to introduce the fluid to the catheter when the suction source is not actively applying the suction force to the catheter.

Example 45: The medical aspiration system of any one of examples 40-43, wherein the control circuitry is configured to control at least one of the fluid source or the fluid control valve to introduce the fluid to the catheter when the suction source is actively applying the suction force to the catheter.

Various aspects of the disclosure have been described. These and other aspects are within the scope of the following claims.

What is claimed is:

1. A medical system comprising:
a catheter having a proximal end and a distal end and configured to traverse vasculature of a patient;
a suction source configured to apply a suction force to the catheter to aspirate a material proximate the distal end of the catheter and from within the vasculature of the patient; and
a fluid controller configured to control an introduction of a fluid into the catheter between the proximal and distal ends to reduce an amount of blood aspirated from a blood vessel of the vasculature of the patient through the catheter.

2. The medical system of claim 1, wherein the system is configured to displace blood aspirated into the catheter through an opening at or near the distal end by the introduction of the fluid into the catheter.

3. The medical system of claim 1, further comprising control circuitry configured to control the suction force at a distal opening of the catheter and control the fluid controller to reduce the amount of blood aspirated from the blood vessel relative to an amount of blood aspirated from the blood vessel without the introduction of the fluid.

4. The medical system of claim 1, further comprising:
an aspiration valve configured to control an amount of the suction force applied by the suction source to the catheter; and
control circuitry configured to control the fluid controller to increase a rate at which the fluid is introduced into the catheter prior to controlling the aspiration valve to increase the suction force at a distal opening of the catheter.

5. The medical system of claim 1, further comprising control circuitry configured to control the fluid controller to introduce the fluid to the catheter when the suction source is actively applying the suction force to the catheter.

6. The medical system of claim 1, further comprising control circuitry configured to control the fluid controller to introduce the fluid to the catheter when the suction source is not actively applying the suction force to the catheter.

7. The medical system of claim 1, wherein the suction source is configured to apply the suction force to a lumen of the catheter, the medical system further comprising:
a fluid source configured to introduce the fluid into the lumen via the fluid controller; and
control circuitry configured to control the suction source, the fluid source, and the fluid controller to alternate the application of the suction force to the catheter and the introduction of the fluid into the lumen.

8. The medical system of claim 1, further comprising:
an aspiration valve configured to control the amount of the suction force applied by the suction source to the catheter; and
control circuitry configured to control at least one of the suction source or the aspiration valve to apply the suction force to the catheter in one or more pulses, wherein the suction force is configured to remove a first volume of blood from the patient via the catheter during the one or more pulses, and wherein the control circuitry is configured to control at least one of the fluid source or the fluid controller to introduce a second volume of the fluid to the catheter between one or more of the one or more pulses.

9. The medical system of claim 8, wherein the suction source is configured to aspirate a thrombus through a lumen of the catheter in a first flow direction, wherein the fluid source is configured to introduce the fluid to the lumen in a second flow direction opposite the first flow direction, and wherein the second volume is equal to or greater than the first volume, and wherein the second volume of the fluid displaces the first volume of the blood in the second flow direction.

10. The medical system of claim 1, further comprising control circuitry configured to control the fluid controller to introduce the fluid to the catheter based on at least one of determining that the catheter has not captured a thrombus, determining a flow rate of a body fluid from the patient through the catheter is greater than or equal to a threshold flow rate, or determining a pressure within the catheter is equal to or greater than a threshold pressure.

11. The medical system of claim 1, further comprising control circuitry configured to control the fluid controller to stop introducing the fluid to the catheter based on at least one of determining that the catheter has captured a thrombus, determining a flow rate of a body fluid from the patient through the catheter that is less than a threshold flow rate, or determining a pressure within the catheter is less than a threshold pressure.

12. A method comprising:
controlling, by control circuitry, a suction source to apply a suction force to a catheter to aspirate a material at a distal opening of the catheter and from within vasculature of a patient; and
controlling, by the control circuitry, a fluid controller to introduce a fluid into the catheter between proximal and distal ends of the catheter to reduce an amount of blood aspirated from a blood vessel of the patient through the catheter.

13. The method of claim 12, wherein controlling the fluid controller to introduce the fluid comprises controlling the fluid controller to introduce the fluid into the catheter to displace blood aspirated into the catheter through an opening at or near the distal end of the catheter.

14. The method of claim 12, wherein controlling the suction source comprises controlling, by the control circuitry, an aspiration valve to control an amount of the suction force applied by the suction source at the distal opening of the catheter,
wherein controlling the aspiration valve and the fluid controller reduces the amount of blood aspirated from the blood vessel relative to an amount of blood aspirated from the blood vessel without the introduction of the fluid.

15. The method of claim 12, further comprising controlling, by the control circuitry, the fluid controller to increase a rate at which the fluid is introduced into the catheter prior to controlling the aspiration valve to increase the suction force at the distal opening of the catheter.

16. The method of claim 12, wherein controlling the fluid controller to introduce the fluid into the catheter comprises controlling the fluid controller to introduce the fluid into the catheter when the suction source is not actively applying the suction force to the catheter.

17. The method of claim 12, wherein controlling the fluid controller to introduce the fluid into the catheter comprises controlling the fluid controller to introduce the fluid into the catheter when the suction source is actively applying the suction force to the catheter.

18. The method of claim 12, wherein a fluid source is configured to introduce the fluid into the lumen via the fluid controller, and wherein controlling the suction source to apply the suction force to the catheter and controlling the fluid controller to introduce the fluid into the catheter comprises controlling, by the control circuitry, the suction source and the fluid source to alternate the application of the suction force to the catheter and introducing the fluid to the catheter.

19. The method of claim 12, wherein controlling the suction source to apply the suction force to the catheter comprises controlling, by the control circuitry, at least one of the suction source or an aspiration valve to apply the suction force to the catheter in one or more pulses, wherein the suction force is configured to remove a first volume of blood from the patient via the catheter during the one or more pulses, and
wherein controlling the fluid controller to introduce the fluid into the catheter comprises controlling at least one of a fluid source or the fluid controller to introduce a second volume of the fluid to the catheter between one or more of the one or more pulses.

20. The method of claim 19, wherein the suction source is configured to aspirate a thrombus through a lumen of the catheter in a first flow direction, wherein the fluid source is configured to introduce the fluid to the lumen in a second flow direction opposite the first flow direction, and wherein the second volume is equal to or greater than the first volume, and wherein the second volume of the fluid displaces the first volume of the blood in the second flow direction.

21. The method of claim 12, further comprising:
determining, by the control circuitry, that at least one of the catheter has not captured a thrombus, a flow rate of a body fluid from the patient through the catheter is greater than or equal to a threshold flow rate, or a pressure within the catheter is greater than or equal to a threshold pressure; and
controlling, by the control circuitry, the fluid controller to introduce the fluid based on determining that at least one of the catheter has not captured the thrombus, the flow rate of the body fluid through the catheter is greater than or equal to the threshold flow rate, or the pressure within the catheter is greater than or equal to the threshold pressure.

22. The method of claim 12, further comprising:
determining, by the control circuitry, that at least one of the catheter has captured a thrombus, a flow rate of a body fluid from the patient through the catheter is less than a threshold flow rate, or a pressure within the catheter is less than a threshold pressure; and
controlling, by the control circuitry, the fluid controller to introduce the fluid based on determining that at least one of the catheter has captured the thrombus, the flow rate of the body fluid through the catheter is less than the threshold flow rate, or the pressure within the catheter is less than the threshold pressure.

23. A medical aspiration system comprising:
a catheter defining a catheter lumen;
a suction source configured to apply a suction force to the catheter lumen to remove a thrombus from a blood vessel of a patient via the catheter lumen;
a fluid source configured to supply a fluid to the catheter lumen in a distal direction to displace blood in the catheter lumen;
a fluid control valve configured to control an introduction of a fluid into the catheter lumen; and
control circuitry configured to control at least one of the suction source, the fluid source, or the fluid control valve to cycle between applying the suction force to the catheter lumen and introducing the fluid into the catheter lumen, wherein introducing the fluid into the catheter lumen displaces an amount of blood into the blood vessel of the patient and reduces a volume of blood aspirated from the blood vessel through the catheter lumen.

24. The medical aspiration system of claim 23, wherein the control circuitry is configured to control the at least one of the suction source, the fluid source, or the fluid control valve to cycle between applying the suction force to the catheter lumen and introducing the fluid into the catheter lumen by at least controlling the suction source or an aspiration valve to apply the suction force to the catheter in one or more pulses and controlling at least one of the fluid source or the fluid control valve to introduce the fluid to the catheter between one or more of the one or more pulses.
